(19) United States
(12) Patent Application Publication
(10) Pub. No.: US 2007/0050165 A1
(43) Pub. Date: Mar. 1, 2007

(54) System and method for automatic self-alignment of a surgical laser

(75) Inventors: Gary P. Gray, Orlando, FL (US); John J. Bowes, Orlando, FL (US); Ricardo Omori, Oviedo, FL (US)

Correspondence Address: ALCON, IP LEGAL, TB4-8, 6201 SOUTH FREEWAY, FORT WORTH, TX, 76134, US

(73) Assignee: Alcon RefractiveHorizons, Inc.

(21) Appl. No.: 11/510,909

(22) Filed: Aug. 28, 2006

Related U.S. Application Data
(60) Provisional application No. 60/713,201, filed on Aug. 31, 2005.

(51) Int. Cl. G01M 19/00; G01D 3/00; G06F 19/00; G01P 21/00; G01R 35/00; A61F 9/008

(52) U.S. Cl. 702/108; 606/10

(57) ABSTRACT

Embodiments of the present invention provide an alignment system operable to align a laser associated with a laser vision correction system. One embodiment of the alignment system comprises a laser source, a beam steering device, a system controller, a partially reflective surface, focusing optics, and an optical detector. The laser source generates a laser beam that the beam steering device receives and redirects along either a surgical pathway or an alignment pathway. The system controller couples to the beam steering device and directs the beam steering device to choose which pathway to be utilized. Additionally, the system controller may control the pulse repetition rate, intensity, beam profile and alignment of the laser beam. The partially reflective surface, within the alignment pathway, partially reflects the laser beam towards alignment coordinates on the surface of the optical detector. Focusing optics, if needed, may further be utilized to focus the partially reflected laser beam on the surface of the optical detector. The optical detector senses the coordinates associated with the illuminated portion of the surface of the optical detector. The system controller compares the coordinates associated with the alignment coordinates and the sensed coordinates and generates a position offset which is used to align the laser beam/steering device.

### CROSS-REFERENCE TO RELATED APPLICATIONS

This application claims priority under 35 U.S.C. §119 to U.S. Provisional Patent Application No. 60/713,201, filed Aug. 31, 2005, the entire contents of which are incorporated herein by reference.

### TECHNICAL FIELD OF THE INVENTION

The present invention relates generally to lasers, and more particularly, to surgical lasers. Even more particularly, the present invention relates to a system and method for aligning a laser beam.

### BACKGROUND OF THE INVENTION

The application of lasers to vision correction has opened new possibilities for treating nearsightedness, farsightedness, astigmatism, and other conditions of the eye. Specifically, laser technology has allowed the development of modern laser techniques that are collectively known as laser vision correction.

These laser vision correction techniques can be used to reshape the surface or subsurface of an eye 10, as shown in FIG. 1. These techniques may employ a cool beam of light (such as Excimer laser beam 12) to remove microscopic amounts of tissue. The removal of this tissue changes the shape of cornea 14 to allow sharper focusing of images and reducing a patient's dependence on glasses and/or contact lenses. Laser vision corrective surgeries include, but are not limited to, laser-assisted in situ keratomileusis (LASIK), laser epithelial keratomileusis (LASEK), epi-LASIK, automated lamellar keratoplasty (ALK), photo ablation procedures such as photo refractive keratectomy (PRK), and other like procedures.

In these procedures, the quality of the results of the laser vision correction may depend upon the ability of the laser 12 to precisely remove tissue from the surface or beneath the surface of cornea 14. Accurately removing tissue with laser 12, in turn may at least in part depend on the ability to accurately align and control the laser.

Laser vision correction systems may employ galvanometers to direct the laser energy to specific locations within eye 10. Galvanometers or other like scanning mechanisms, however, may experience temperature-induced drift, which, if not adequately compensated for, may adversely affect the accuracy of positioning the laser beam at desired locations within the patient's eye. Thermal fluctuations in the laser cavity may induce “beam wander” resulting in a similar effect.

One of the most time consuming portions of a laser vision correction procedure is the set up and alignment of the surgical laser. Existing laser vision correction systems typically employ manual techniques to align the laser prior to the laser vision correction procedure. Further, laser vision correction procedures often require re-alignment of the laser between patients or even between performing the procedure on an individual patient's two eyes.

Laser misalignment is typically compensated for by directing the laser at known locations where video or image processing is used to produce an offset which can then be applied to the laser. This position offset typically requires a manual alignment of the laser. Additionally, this method works best for linear translators that have minimal thermal drift. However, when using galvanometers, it is advantageous to more frequently perform an alignment procedure and to minimize elapsed time between alignment and treatment.

Therefore, there is a need for a system and method for automatic self-alignment of a laser, such as a surgical laser, that can reduce or eliminate the above-stated problems of prior art laser alignment systems and methods.

### SUMMARY OF THE INVENTION

The embodiments of the system and method for automatic self-alignment of a laser of the present invention substantially meet these needs and others. Embodiments of the present invention may provide an alignment system operable to align a laser, such as an excimer laser associated with a laser vision correction system. An embodiment of the alignment system of this invention can comprise a laser source, a beam steering device, a system controller, a partially reflective surface, focusing optics, and an optical detector. The laser source generates a laser beam, such as an excimer laser beam, that the beam steering device is operable to receive and redirect along either a surgical pathway or an alignment pathway. The system controller couples to the beam steering device and is operable to direct which pathway the beam steering device selects. Additionally, the system controller may control the pulse repetition rate, intensity, beam shape and alignment of the laser beam, and/or perhaps other such laser parameters. The partially reflective surface located within the alignment pathway partially reflects the laser beam towards alignment positions on the surface of an optical detector. Additionally, optional focusing optics may be utilized to focus the partially reflected laser beam on the surface of the optical detector. The optical detector is operable to sense the coordinates associated with a portion of the surface of the optical detector illuminated by the partially reflected laser beam. The system controller then can compare the coordinates associated with alignment positions and the sensed coordinates to generate a position offset. The position offset can then be applied to align the beam steering device.

Another embodiment of the present invention can provide a method of aligning a laser beam used in laser vision correction. This method involves directing the laser beam at a partially reflective surface. The laser beam partially reflected from the partially reflective surface is directed at alignment coordinates on a surface of an optical detector. The transmitted portion of the laser beam (i.e., that portion not reflected by the partially reflective surface) is received by an optical beam dump. The partially reflective laser beam can be focused on the surface of the optical detector to illuminate a portion of the surface of the optical detector. The optical detector senses the coordinates of the actually illuminated areas of the optical detector. These sensed actual illuminated coordinates are compared with the alignment coordinates to produce a position offset. This position offset is then used to align the laser beam. The position offset may be in the form of (x,y) offsets at the surface or angular displacements associated with the alignment pathway.

Another embodiment of the present invention can provide a laser vision correction surgical system. This laser vision correction surgical system can comprise a laser source and beam steering device that are both directed by a system controller. Additionally, an alignment system may be used to align the laser beam. The alignment system, similar to that of previously discussed embodiments, comprises a beam steering device, a partially reflective surface, focusing optics, and an optical detector. The system controller is operable to cause the beam steering device to direct the laser beam along an alignment pathway to specific coordinates on the surface of the optical detector. The alignment pathway between the beam steering device and optical detector may include a partially reflective surface operable to partially reflect the laser beam. Partial reflection reduces the intensity of the laser beam seen by the optical detector. Focusing optics in the alignment pathway optionally focus the partially reflected laser beam on the surface of the optical detector. The system controller compares coordinates associated with the illuminated portion of the optical detector with the coordinates that the laser beam has been directed to in order to generate a position offset. This position offset is then used to align the laser beam.

The above described embodiments of the present invention, as well as other embodiments, facilitate the alignment of a laser beam within a laser vision correction system. By facilitating the alignment of the laser beam, the embodiments of this invention may also simplify the set up of the surgical laser, such as an excimer laser, and help reduce the setup time for a surgical procedure. Additionally, the embodiments of the present invention facilitate more frequent alignment of the laser beam, thus allowing laser vision correction procedures to be performed more accurately and in less time.

### BRIEF DESCRIPTION OF THE DRAWINGS

For a more complete understanding of the present invention and the advantages thereof, reference is now made to the following description taken in conjunction with the accompanying drawings in which like reference numerals indicate like features and wherein:

FIG. 1 illustrates a laser vision correction procedure where an excimer laser beam is used to reshape a patient's cornea;

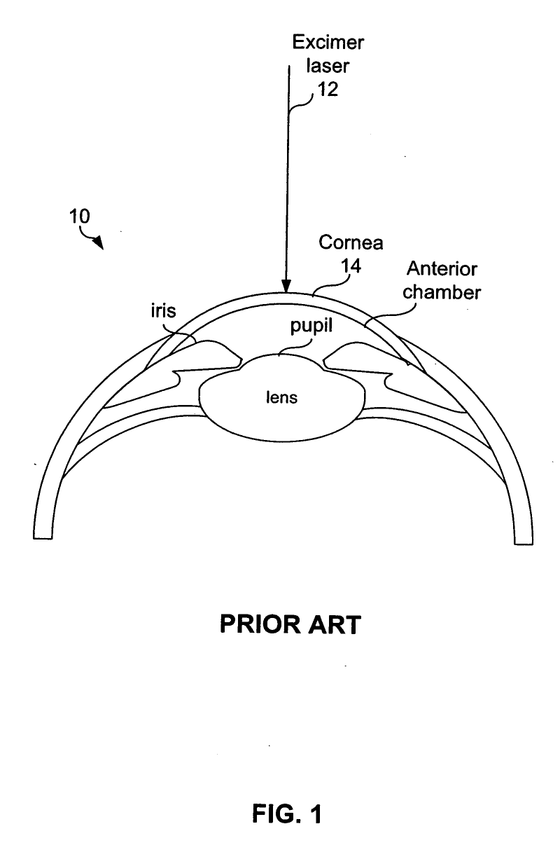

FIG. 2 is a functional diagram of a basic alignment optical setup in accordance with an embodiment of the present invention;

FIG. 3 illustrates the determination of a position offset associated with the specified alignment coordinates and actual illuminated coordinates in accordance with an embodiment of the present invention;

FIG. 4 is a functional block diagram of an optical setup in accordance with an embodiment of the present invention; and

FIG. 5 is a logic flow diagram illustrating the steps of one embodiment of an alignment method according to the present invention.

### DESCRIPTION OF THE INVENTION

Preferred embodiments of the present invention are illustrated in the FIGUREs, like numerals being used to refer to like and corresponding parts of the various drawings.

Embodiments of the present invention substantially address the problem of laser beam misalignment associated with a refractive vision correction procedure performed using a laser, such as an excimer laser. For example, thermal drift associated with galvanometer scanner systems used to position a surgical laser beam can effectively misalign the treatment beam used for such a procedure. Misalignment of the treatment laser beam would likely result in what is referred to as a “de-centered ablation”. Angular drift within a laser itself produces a similar phenomenon. Embodiments of the present invention address both galvanometer and laser drift by providing for automatic alignment of the origin location of a laser vision correction system using a quad-cell, CCD or other like optical detector.

In performing refractive surgery, there are several approaches for directing the energy of a laser to a desired location within an eye. Current techniques include galvanometer scanning, linear translation using “voice coil” type motors, and moving apertures. Embodiments of the present invention specifically address limitations associated with galvanometer scanning as it is applied to refractive surgery.

Galvanometers are susceptible to temperature-induced drift which, if not adequately compensated for, may adversely affect the accuracy of positioning a laser beam during refractive surgery or other laser vision correction. Thermal fluctuations in the laser cavity may also induce beam wander that can result in a similar effect. Embodiments of the present invention can reduce or eliminate aim-point error by “homing” the laser beam using an internal optical detector. Such auto alignment may be performed just prior to an ablation procedure. The time between compensation and application of the treatment can thus be reduced.

Additionally, thermal fluctuations in the scanning subsystem of a surgical laser system may be reduced by employing an “ideal pattern” which in most respects can simulate the motion and pulse repetition characteristics of actual surgical procedures. This ideal pattern, which runs whenever the system is not executing an ablation procedure, can contain similar moves and travel requirements as are required in actual surgical patterns. By implementing this procedure, the thermal stability of the surgical system between idle periods and actual surgical proceedings can be greatly enhanced.

The energy emitted by an excimer laser is typically enough to damage a quad-cell detector. However, the embodiments of the present invention direct only a small portion of the radiated excimer laser energy towards the detector. This effect is achieved with the use of a partially reflective surface. Alternatively, the wavelength of the excimer laser beam may be shifted to prevent damage to the optical detector.

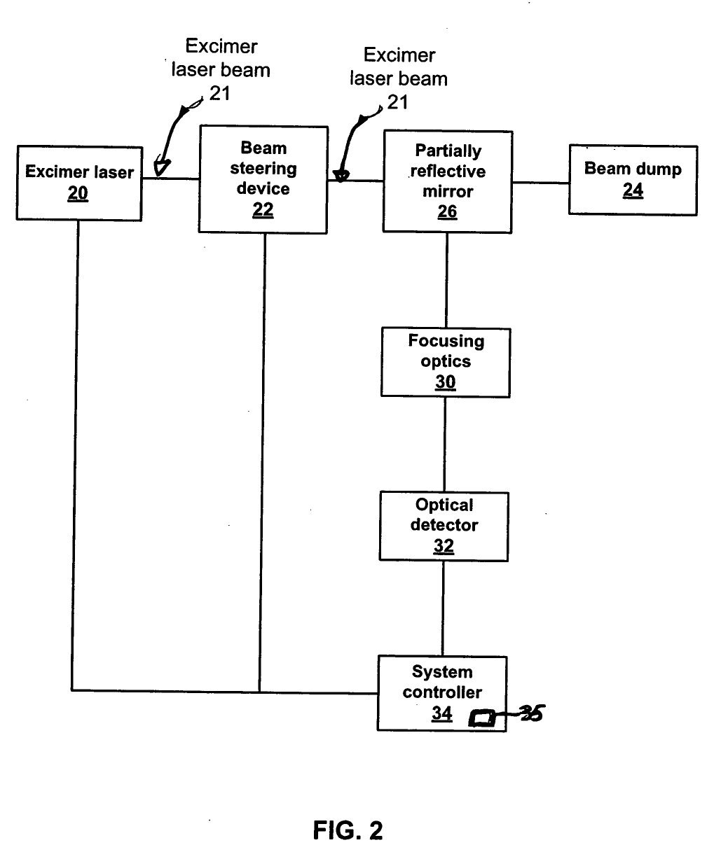

FIG. 2 is a functional diagram of a basic alignment optical setup in accordance with an embodiment of the present invention. This optical setup includes laser source 20, which can be an excimer laser, beam steering device 22, beam dump 24, partially reflective surface 26, focusing optics 30, optical detector 32, and system controller 34. Laser source 20 produces a laser beam 21 which is supplied to the beam steering device 22. System controller 34 provides position and position offset commands to the beam steering device 22. To prevent damage to optical detector 32, partially reflective mirror 26 directs only a small portion of the laser beam 21 energy from beam steering device 22 along the alignment pathway towards optical detector 32. The remaining laser beam 21 energy is directed elsewhere; for example, to optical beam dump 24.

System controller 34 may be a single processing device or a plurality of processing devices. Such a processing device may be a microprocessor, microcontroller, digital signal processor, microcomputer, central processing unit, field programmable gate array, programmable logic device, state machine, logic circuitry, analog circuitry, digital circuitry, and/or any device that manipulates signals (analog and/or digital) based on operational instructions stored in a memory, such as memory 35. The memory may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the system controller implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The memory 35 stores, and the system controller 34 executes, operational instructions corresponding to at least some of the steps and/or functions illustrated in FIGS. 2-5.

Optical detector 32 may be a quad-cell detector, charge-coupled device (CCD), or other known light-sensitive or image sensor. A CCD detector, when compared to a quad-cell, allows improved resolution of the illuminated coordinates on the surface of the detector. This improved resolution results in an improved alignment capability of the laser beam. The use of a CCD, as opposed to a quad-cell detector, in addition to adding a finer resolution may also allow beam profiling of the beam as detected by optical detector 32 to be performed.

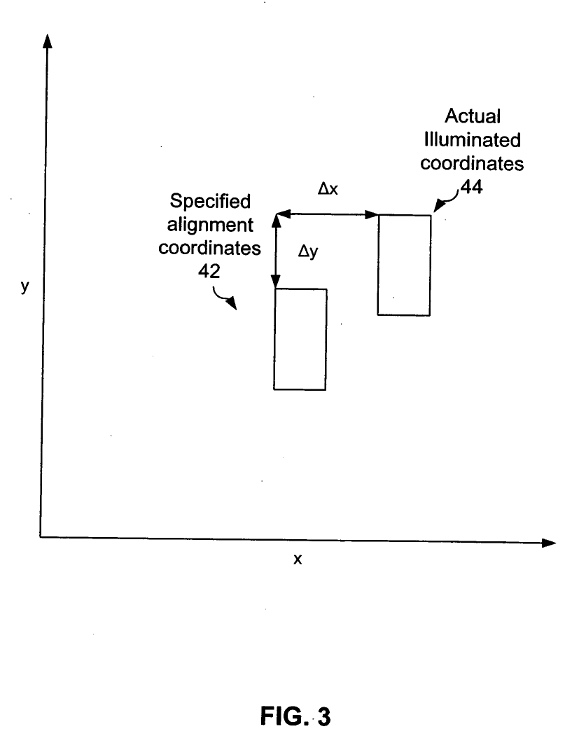

FIG. 3 illustrates how such an optical detector 32 may be used to generate a position offset with which to align the laser 20. In FIG. 3, specified alignment coordinates 42 are identified as (x,y) coordinates on the surface of optical detector 32. A predetermined beam profile may also be associated with these coordinates. Similarly, the actual illuminated coordinates 44 may also be described as a set (x,y) of coordinates on the surface of optical detector 32. This set of actual coordinates may also be used to describe an actual beam profile. The actual illuminated coordinates 44 may be compared with the specified alignment coordinates 42 to produce an x and y offset shown as (Δx,Δy). This position offset may then be applied to the beam steering device 22 in order to align laser beam 21 such that the actual illuminated coordinates 44 align (correspond) to the specified alignment coordinates 42. Beam profiling comparisons allow the actual beam profile to be manipulated to match that of the desired beam profile. The alignment reduces position errors and set up time associated with preparing the laser beam for a laser vision corrective procedure and can improve the overall accuracy of laser vision correction techniques employing such lasers. Additionally, by automating this alignment process, the time between alignment and actual performance of the laser vision correction can be reduced. This allows alignments to be performed more frequently and can make it practical to perform alignments between laser vision correction applications of both eyes of an individual patient.

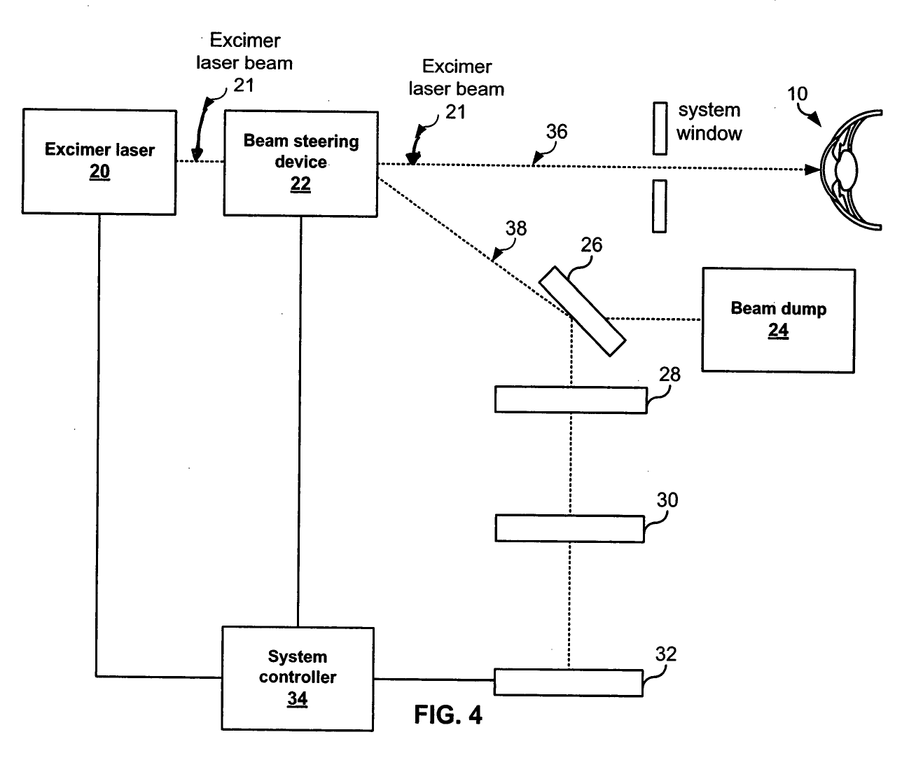

FIG. 4 is a functional diagram of an optical set up according to one embodiment of this invention that is similar to that of the optical set up described with reference to FIG. 2. FIG. 4 adds fluorescent material 28 in the alignment pathway. Fluorescent material 28, or other like means, converts the invisible UV radiation of the laser to a desired wavelength within the detection range of optical detector 32. To achieve an adequate signal to noise ratio (SNR), focusing optic 30 may be included to image the fluorescent output from fluorescent material 28 onto the active areas of the optical detector 32. The florescent material 28 may also allow for image persistence on optical detector 32, if needed.

As shown in FIG. 4, the alignment pathway is off axis from the surgical pathway. Alignment pathway 38 comprises approximately the same linear distance as surgical pathway 36. These pathways constitute the distance from beam steering device 22 to optical detector 32 and to the plane of the patient's eye 10, respectively.

To reduce the intensity of radiated energy directed along the alignment pathway 38, a partially reflective mirror or surface 26 may be employed. In one embodiment, a 5 percent reflective mirror is used. Thus, in the embodiment of FIG. 4, for example, the remaining 95 percent of the laser beam 21 energy incident on reflective mirror 26 is directed to a beam dump 24.

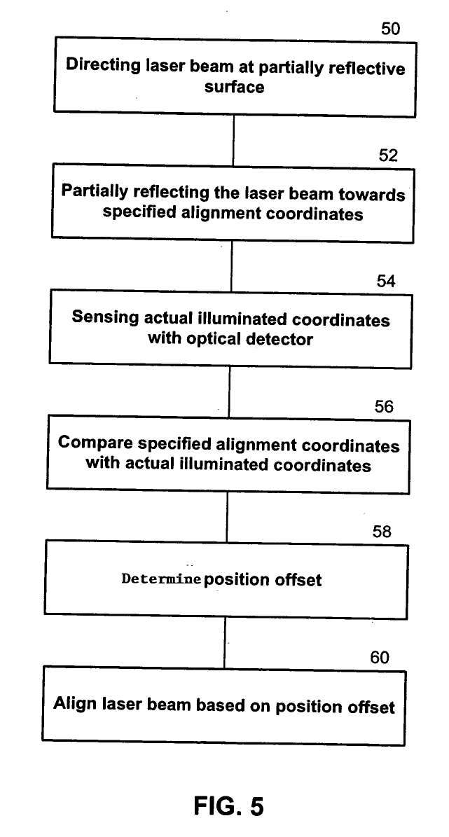

FIG. 5 is a logic flow diagram illustrating the steps of one embodiment of an alignment method according to the present invention. In step 50, a laser beam, which can be an excimer laser beam or a beam from another like laser used in laser vision correction, is directed along an alignment pathway and towards a partially reflective surface. The partially reflective surface reduces the intensity of the portion of the laser beam used to align the laser beam. This will prevent inadvertent damage to the optical detector used to align the laser beam. In step 52, the partially reflected laser beam is directed toward alignment coordinates on the optical detector. The alignment coordinates are specified coordinates on the surface of the optical detector. The partially reflected laser beam will be incident on and illuminate actual coordinates on the optical detector, which may or may not correspond to the alignment coordinates. The actual coordinates associated with the illuminated portion of the optical detector are sensed and identified at step 54. At step 56, the specified alignment coordinates and the actual illuminated coordinates are compared to one another. The difference between these coordinates corresponds to a position offset, determined at step 58. Because the alignment pathway and the surgical pathway (the pathway of the laser beam directed towards a patient's eye) are about equal, the position offset produced by this method may be used directly to align the laser beam in step 60. Other embodiments of the present invention may determine an angular offset associated with the difference in the specified alignment coordinates and the actual illuminated coordinates. Such an embodiment would allow greater variation between the alignment pathway and surgical pathway. The difference between the illuminated coordinates and the specified coordinates is detected by the optical detector, which can be, for example, a quad cell detector or CCD detector, or through the use of video and/or image processing.

Embodiments of the present invention can be used to align the laser vision correction laser beam between patients or between procedures associated with an individual patient. For example, the laser beam may be aligned between performing a procedure on a patient's first eye and performing the procedure on the patient's second eye. Other circumstances may arise that require the realignment of the laser vision correction laser beam, such as a change in the pulse repetition rate of the laser. Embodiments of the present invention provide the ability to align a surgical or other laser at the frequency with which a laser vision correction procedure is to be performed.

Other situations may also require laser beam alignment. For example, after exposing positioning devices, such as galvanometers, to thermal changes, it may be desirable to realign or verify the alignment of the laser vision correction laser prior to the performance of any additional surgical procedures. To prevent or reduce the thermal transient effects of laser heating, the laser vision correction laser may be placed in an idle mode or loop such that the surgical pathway is directed toward a beam dump when the laser is not employed in performance of a procedure. This will minimize thermal transients associated with heating and cooling of the beam steering device when utilized to actually steer the laser.

Embodiments of the present invention advantageously provide an accurate and repeatable alignment mechanism and alignment method. The time required to align or otherwise calibrate a laser between patients can thus be greatly reduced or eliminated as compared to prior art manual geometry adjustments or other like calibrations. For example, this reduced setup time allows alignment to be performed between eyes of a bilateral case without an additional time penalty.

Additionally, the embodiments of the present invention may be used to automatically compensate for system misalignments from a variety of sources without requiring external mechanisms. Other aspects of the present invention may help maintain a stable operating temperature within the beam scanning mechanism in order to further reduce fluctuations in system performance.

As one of average skill in the art will appreciate, the term “substantially” or “approximately”, as may be used herein, provides an industry-accepted tolerance to its corresponding term. Such an industry-accepted tolerance ranges from less than one percent to twenty percent and corresponds to, but is not limited to, component values, integrated circuit process variations, temperature variations, rise and fall times, and/or thermal noise. As one of average skill in the art will further appreciate, the term “operably coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. As one of average skill in the art will also appreciate, inferred coupling (i.e., where one element is coupled to another element by inference) includes direct and indirect coupling between two elements in the same manner as “operably coupled”. As one of average skill in the art will further appreciate, the term “compares favorably”, as may be used herein, indicates that a comparison between two or more elements, items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

Although the present invention is described in detail, it should be understood that various changes, substitutions and alterations can be made hereto without departing from the spirit and scope of the invention as described.

What is claimed is:

1. A method of aligning a laser beam within a corrective vision surgical system, the method comprising:
directing the excimer laser beam at a partially reflective surface, wherein the partially reflective surface reflects a portion of the laser beam along a path towards specified alignment coordinates on a surface of an optical detector;
focusing the partially reflected portion of the laser beam on the surface of the optical detector, wherein the focused laser beam portion illuminates actual coordinates on the surface of the optical detector;
sensing the location of the actual illuminated coordinates with the optical detector;
comparing the actual illuminated coordinates location and the specified alignment coordinates location to determine a position offset; and
aligning the laser beam using the position offset.

2. The method of claim 1, wherein the optical detector comprises a quad-cell optical detector.

3. The method of claim 1, wherein the optical detector comprises a charge coupled device (CCD).

4. The method of claim 1, wherein the laser beam is an excimer laser beam.

5. The method of claim 1, wherein a length of the path to the surface of the optical detector is about equal to a length of a path to a patient's eye.

6. The method of claim 1, wherein the aligned laser beam is used to perform a laser vision correction procedure.

7. The method of claim 6, wherein the laser vision correction procedure comprises at least one procedure selected from the group consisting of laser-assisted in situ keratomileusis (LASIK), laser epithelial keratomileusis (LASEK), epi-LASIK, automated lamellar keratoplasty (ALK), and photo refractive keratectomy (PRK).

8. The method of claim 1, further comprising altering a wavelength of the partially reflected laser beam.

9. The method of claim 1, further comprising dumping at least a portion of the laser beam not reflected by the partially reflective surface towards the optical detector.

10. A laser vision correction surgical system, comprising:
a laser source operable to generate a laser beam;
a beam steering device operable to direct the laser beam to a patient's eye; and
an alignment system operable to align the laser beam, wherein the alignment system comprises:
a system controller operable to direct the beam steering device to direct the laser beam to at least one alignment position on the surface of an optical detector;
a partially reflective surface within a path of the laser beam and the at least one alignment position, wherein the laser beam is partially reflected towards the at least one alignment position by the partially reflective surface;
focusing optics operable to focus the partially reflected laser beam on the surface of the optical detector, wherein the optical detector is operable to sense coordinates associated with a portion of the surface of the optical detector illuminated by the partially reflected laser beam, and wherein the system controller is operable to compare coordinates associated with the alignment position and the sensed coordinates and generate a position offset based on the comparison, wherein the position offset is applied to the beam steering device to align the laser beam.

11. The laser vision correction system of claim 10, wherein the optical detector comprises a quad-cell optical detector.

12. The laser vision correction system of claim 10, wherein the optical detector comprises a charge coupled device (CCD).

13. The laser vision correction system of claim 10, wherein the laser source is an excimer laser source.

14. The laser vision correction system of claim 10, wherein a length of the path to the surface of the optical detector is about equal to a length of a path to a patient's eye.

15. The laser vision correction system of claim 10, wherein the aligned laser is used to perform a laser vision correction procedure.

16. The laser vision correction system of claim 15, wherein the laser vision correction procedure comprises at least one procedure selected from the group consisting of laser-assisted in situ keratomileusis (LASIK), laser epithelial keratomileusis (LASEK), epi-LASIK, automated lamellar keratoplasty (ALK), and photo refractive keratectomy (PRK).

17. The laser vision correction system of claim 10, further comprising fluorescent optics operable to altering a wavelength of the partially reflected laser beam.

18. The laser vision correction system of claim 10, further comprising a beam dump operable to accept at least a portion of the laser beam not partially reflected towards the at least one alignment position by the partially reflective surface.

19. An alignment system operable to align a laser of a laser vision correction system, the alignment system comprising:
a laser source operable to generate a laser beam;
a beam steering device operable to:
receive the laser beam; and
redirect the laser beam along a surgical pathway or along an alignment pathway;
a system controller operably coupled to the beam steering device, wherein the system controller is operable to direct the beam steering device to select the surgical pathway or alignment pathway;
a partially reflective surface within the alignment pathway, wherein the laser beam is partially reflected towards at least one alignment position on the surface of an optical detector by the partially reflective surface; and
focusing optics operable to focus the partially reflected laser beam on the surface of the optical detector, wherein the optical detector is operable to sense coordinates associated with a portion of the surface of the optical detector illuminated by the partially reflected laser beam, and wherein the system controller is operable to compare coordinates associated with the alignment position and the sensed coordinates and generate a position offset based on the comparison, wherein the position offset is applied to the beam steering device to align the laser beam.

20. The alignment system of claim 19, wherein the optical detector comprises a quad-cell optical detector.

21. The alignment system of claim 19, wherein the optical detector comprises a charge coupled device (CCD).

22. The alignment system of claim 19, wherein the laser source is an excimer laser source.

23. The alignment system of claim 19, wherein a length of the surgical pathway and a length of the alignment pathway are about equal.

24. The alignment system of claim 19, wherein the aligned laser beam is used to perform a laser vision correction procedure.

25. The alignment system of claim 24, wherein the laser vision correction procedure comprises at least one procedure selected from the group consisting of laser-assisted in situ keratomileusis (LASIK), laser epithelial keratomileusis (LASEK), epi-LASIK, automated lamellar keratoplasty (ALK), and photo refractive keratectomy (PRK).

26. The alignment system of claim 19, further comprising fluorescent optics operable to alter a wavelength of the partially reflected laser beam.

27. The alignment system of claim 19, further comprising a beam dump operable to accept at least a portion of the laser beam not partially reflected towards the at least one alignment position by the partially reflective surface.
